(19) United States
(12) Patent Application Publication
(10) Pub. No.: US 2026/0039996 A1
(43) Pub. Date: Feb. 5, 2026

(54) AIR ADSORBENT FOR SPEAKERS AND SOUND EQUIPMENT INCLUDING THE SAME

(71) Applicant: NEXVEL CO., LTD, Yongin-si (KR)

(72) Inventors: Dae Lyun KANG, Suwon-si (KR); Seung Chan LEE, Gwangju-si (KR)

(73) Assignee: NEXVEL CO., LTD, Yongin-si (KR)

(21) Appl. No.: 19/056,818

(22) Filed: Feb. 19, 2025

(30) Foreign Application Priority Data
Aug. 5, 2024 (KR) 10-2024-0104250

(51) Int. Cl. H04R 1/28; A61L 9/16; H04R 1/02

(52) U.S. Cl. CPC H04R 1/288; H04R 1/02; A61L 9/16; A61L 2209/22

(57) ABSTRACT

Provided is an air adsorbent for speakers including: an activated carbon fabric comprising activated carbon fibers manufactured by carbonizing a cellulose fabric containing cellulose fibers, wherein the cellulose fabric is manufactured by weaving bundles of the cellulose fibers, wherein each bundle is provided by twisting the plurality of cellulose fibers, the activated carbon fabric has a thickness of about 0.3 mm to about 0.9 mm, an air permeability of about 60 cm³/cm²/s to about 90 cm³/cm²/s, and a BET-specific surface area of about 1000 m²/g to about 1500 m²/g, and each of the activated carbon fibers has pores, each of which has an average diameter of about 1 nm to about 5 nm.

### CROSS-REFERENCE TO RELATED APPLICATION

This application claims priority to Korean Patent Application No. 10-2024-0104250 filed on Aug. 5, 2024, and all the benefits accruing therefrom under 35 U.S.C. § 119, the contents of which are incorporated by reference in their entirety.

### BACKGROUND

The present disclosure relates to an air adsorbent for speakers and sound equipment including the same. An air adsorbent according to one embodiment is suitable for speaker-box systems included in various devices such as televisions and automobiles.

In recent micro speaker-box systems used in smartphones, a micro-porous zeolite granule air adsorbent (hereinafter referred to as an air adsorbent), which has a volumetric expansion effect through micropores several nanometers in size, is utilized to achieve enhanced low-frequency reproduction even in a compact back volume of 1 cc or less.

Meanwhile, in speaker-box systems equipped with medium-to-large speakers with an output of several tens of watts for televisions, the thickness of the speaker-box systems is being reduced to 10 mm or less as televisions themselves become thinner. As a result, unlike before, it is no longer possible to create a large back volume of several hundred cubic centimeters, which was used to achieve excellent acoustic reproduction characteristics in the low-frequency range.

To overcome these limitations, high-end ultra-thin TV slim speaker-box systems employ a back volume that is both thin and large in volume (with a thickness of 5 mm or less and a volume of 100 cc or more) and enhance the electromagnetic performance of the speaker unit by using a permanent magnet with a high BH(max) or a large volume. This improves the sound pressure level (SPL) in the low-frequency reproduction range of 100 Hz to 300 Hz. Nevertheless, in speaker-box systems with further reduced thickness and an even smaller back volume, applying such technology becomes challenging. Therefore, it is essential to further enhance the SPL in the low-frequency range by using air-adsorbing materials with nanometer-scale micropores, such as zeolite granules or activated carbon. However, filling a volume of several tens to hundreds of cubic centimeters with zeolite granules or activated carbon, which have particle diameters in the micrometer range or smaller, not only excessively increases costs but also poses a problem of degrading sound pressure level characteristics due to impaired airflow of air particles caused by the movement of packed particles within the large back volume.

### PRIOR ART DOCUMENT

### Patent Document

(Patent Document) Korean Patent Registration No. 10-2155642.

### SUMMARY

The present disclosure provides an air adsorbent capable of improving sound quality and sound equipment including the same.

Additionally, the sound equipment of the present invention may improve low-frequency characteristics in speaker-box systems with a large back volume and high output.

Furthermore, an air adsorbent of the present invention may effectively reduce foreign substances such as dust.

In accordance with an exemplary embodiment, an air adsorbent for a speaker includes: an activated carbon fabric comprising activated carbon fibers manufactured by carbonizing a cellulose fabric containing cellulose fibers, In one embodiment, the air adsorbent may further include a pouch member that encloses the activated carbon fabric.

The cellulose fabric may be manufactured by weaving bundles of the cellulose fibers, wherein each bundle is provided by twisting the plurality of cellulose fibers,

The activated carbon fabric may have a thickness of about 0.3 mm to about 0.9 mm, an air permeability of about 60 cm³/cm²/s to about 90 cm³/cm²/s, and a BET-specific surface area of about 1000 m²/g to about 1500 m²/g.

The activated carbon fabric may have pores, each of which has an average diameter of about 1 nm to about 5 nm.

The pouch member may have an air permeability of about 200 cm³/cm²/s to about 550 cm³/cm²/s and a thickness of about 0.07 mm to about 0.15 mm.

In one embodiment, the air adsorbent may include the above-described activated carbon fabric, and an adhesive member configured to adhere surfaces of the activated carbon fabric to maintain a predetermined shape,

The activated carbon fabric may have a roll shape with a hollow defined by being wound at least once.

The activated carbon fabric may include one end providing an inner surface of the roll shape and the other end providing an outer surface of the roll shape, and the adhesive member is disposed on an inner surface of the other end to allow the other end of the activated carbon fabric to adhere to another portion of the activated carbon fabric to be fixed.

In accordance with another exemplary embodiment, sound equipment includes the air adsorbent for the speaker.

In accordance with another exemplary embodiment, a method for manufacturing an air adsorbent for a speaker includes: preparing the activated carbon fabric; rolling the activated carbon fabric so that one end of the activated carbon fabric is disposed on an inner surface thereof to form a hollow roll shape; fixing the other end of the activated carbon fabric using an adhesive member; and cutting the roll-shaped activated carbon fabric along a longitudinal direction of the other end of the activated carbon fabric. After the cutting of the roll-shaped activated carbon fabric: the method may further include inserting the cut roll-shaped activated carbon fabric into an air-permeable pouch; and sealing the air-permeable pouch.

### BRIEF DESCRIPTION OF THE DRAWINGS

Exemplary embodiments can be understood in more detail from the following description taken in conjunction with the accompanying drawings, in which:

FIG. 1 is a view illustrating an air adsorbent of manufacturing example 1 in the form of an activated carbon fabric.

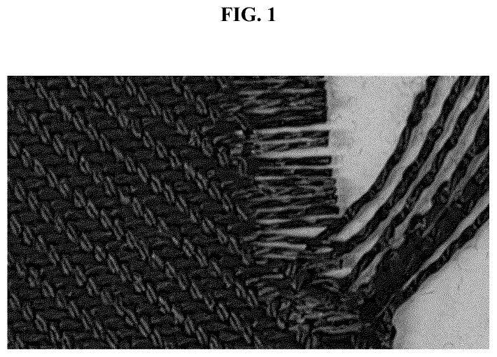

FIG. 2 is a view illustrating an air adsorbent of manufacturing example 4 in the form of an activated carbon nonwoven fabric.

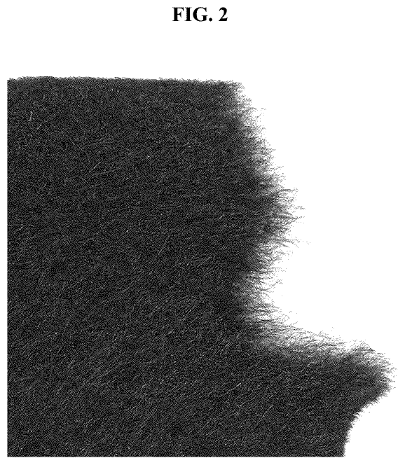

FIG. 3 illustrates a part of the method for manufacturing an air adsorbent, wherein a view (A) illustrates a process of manufacturing an activated carbon fabric into a roll shape, a view (B) illustrates a state in which an adhesive member is attached to an activated carbon fabric, and a view (C) illustrates the activated carbon fabric manufactured into a roll shape, a view (D) is a cross-sectional view of the roll-shaped activated carbon fabric, and a view (E) illustrates a cut activated carbon fabric with a hollow cylindrical roll shape.

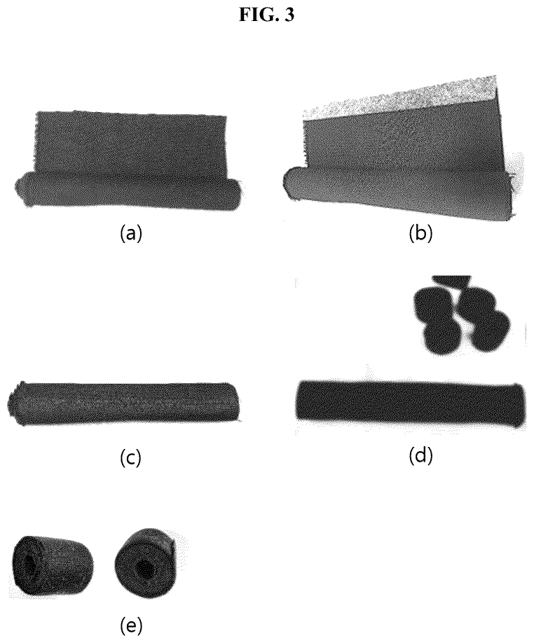

FIG. 4 illustrates an air adsorbent manufactured by inserting the cut activated carbon fabric of the view (E) of FIG. 3 into a pouch member, wherein a view (A) illustrates the use of nonwoven fabric as the pouch member, and a view (B) illustrates the use of a PET shrink film with micro air-permeable holes as the pouch member.

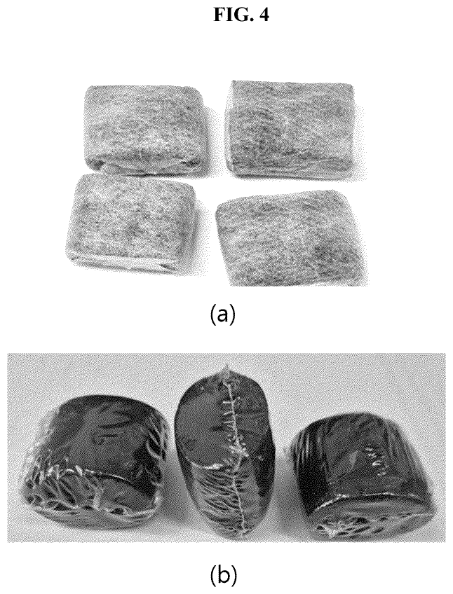

### DETAILED DESCRIPTION OF EMBODIMENTS

Hereinafter, preferred embodiments of the present invention will be described with reference to the accompanying drawings as follows. However, the embodiments of the present invention may be modified in various ways, and the scope of the present invention is not limited to the embodiments described below. Additionally, the embodiments of the present invention are provided to further fully describe the present invention to those of ordinary skill in the art.

### Air Adsorbent for Speakers and Sound Equipment Including the Same

Sound equipment, according to an embodiment of the present invention may include an air-adsorbent for speakers. The air adsorbent for speakers may include an activated carbon fabric that includes activated carbon fibers, which are manufactured by carbonizing a cellulose fabric containing cellulose fibers. In one embodiment, the air adsorbent may further include a pouch member that encloses the activated carbon fabric.

The sound equipment may refer to a device that includes a speaker-box system, which may be an apparatus for generating sound, such as a television or an automobile. The speaker-box system may be a system that includes a core speaker within a box or a housing including a sound resonance space. The sound generated from the speaker may resonate within the sound resonance space inside the box or the housing and may be emitted, thereby reducing sound interference and improving sound quality and volume. Especially, the sound resonance space may be a crucial factor in the acoustic characteristics of the low-frequency range. The larger the sound resonance space, the more advantageous the sound output in the low-frequency range becomes, allowing for an extended output bandwidth. The sound resonance space may have a volume of several hundred cubic centimeters, for example, it may range from about 200 to about 250 cubic centimeters. A speaker-box system with such a large volume may have more difficulty improving the low-frequency range compared to a micro speaker-box system, which has a volume of only several to several dozens of cubic centimeters. Since the speaker-box system of the present invention may require a large volume of air adsorbent, airflow, or ventilation, within the sound resonance space should be considered to effectively improve the low-frequency range. For this purpose, the present invention may preferably provide a fabric sheet-type air adsorbent rather than a conventional particle-, granule-, or powder-type air adsorbent.

The air adsorbent for speakers according to an embodiment of the present invention may be provided in the sound resonance space of the speaker-box system to further improve the acoustic characteristics in the low-frequency range. The air adsorbent may adsorb and desorb air molecules to create a virtual back volume, that is, a virtual sound resonance space, thereby achieving a volume expansion effect that extends the physically limited sound resonance space. Through this effect, the acoustic characteristics in the low-frequency range of the speaker-box system may be improved, and the sound output bandwidth may be extended. Here, the low-frequency range may refer to a frequency range of 1000 Hz or lower, and preferably, it may refer to a frequency range of about 100 to about 700 Hz.

In the present invention, the air adsorbent may be made of an activated carbon fabric. The activated carbon fabric may refer to a structure provided by the intersection of activated carbon fibers. In one embodiment, the activated carbon fabric may have a woven shape in which bundles of fibers, made by twisting a plurality of activated carbon fibers, are interlaced. This structure may provide an activated carbon fabric that is both more durable and has a larger specific surface area.

In one embodiment, the activated carbon fabric may be manufactured using twill weaving. The twill weaving may refer to a weaving method in which warp (vertically oriented threads) and weft (horizontally oriented threads) intersect at regular intervals to create a diagonal pattern, resulting in diagonal lines appearing on the fabric surface. Since the activated carbon fabric is provided with a twill weaving structure, the activated carbon fabric may have higher flexibility compared to other fabric shapes and further improve airflow due to the increased intersection of the warp and weft. Through this structure, the air adsorbent may be rolled into a roll shape, allowing air to move more quickly even when a large-volume sound resonance space is filled with the air adsorbent.

The activated carbon fibers may have an average diameter of about 5 μm to about 20 μm and may have pores with an average diameter of about 1 nm to about 5 nm. Each pore may be provided not only on the surface but also inside the fibers.

The activated carbon fabric may have a thickness ranging from about 0.3 mm to about 0.9 mm, preferably from about 0.4 mm to about 0.45 mm. The activated carbon fabric may have air permeability ranging from about 60 cm³/cm²/sec to about 90 cm³/cm²/sec, preferably from about 75 cm³/cm²/sec to about 80 cm³/cm²/sec. Additionally, the activated carbon fabric may have at least 12 N tensile strength in a longitudinal direction, measured according to ISO 13934-2:2014 (Grab method). When the thickness of the activated carbon fabric is too thick, it may cause issues with airflow, that is, air permeability, during the adsorption and desorption of air molecules. When the fabric is too thin, automation of the cutting process may become difficult. Additionally, when the air permeability of the activated carbon fabric is too high, the number of activated carbon fiber strands may decrease, and the volume of micro-pores may significantly reduce. When the air permeability is too low, the adsorption and desorption performance of the air molecules may decrease. Furthermore, due to issues such as tensile strength, particle dispersion, and manufacturing costs, it may be difficult to produce activated carbon fabric with a specific surface area (SSA) greater than about 1800 m²/g.

The activated carbon fabric may be manufactured by carbonizing cellulose fabrics. The cellulose fabric refers to a structure provided by the intersection of cellulose fibers, and the cellulose fibers refer to fibers made from cellulose extracted from plant or wood pulp. In one embodiment, the cellulose fabric may be manufactured by weaving bundles of twisted cellulose fibers, with each bundle made by twisting a plurality of cellulose fibers. Through this, the activated carbon fabric may be manufactured to be more durable with a larger specific surface area.

The cellulose fibers may be natural cellulose fibers, such as cotton or linen, or synthetic cellulose fibers, such as viscose rayon, modal, or lyocell. Preferably, the cellulose fibers may be viscose rayon. Since viscose rayon is soft, has a smooth surface, and has a glossy finish, activated carbon manufactured from this material may have the advantage of having uniform surface characteristics and not crumbling after manufacturing. Furthermore, viscose rayon is easy to manufacture in various thicknesses, making it suitable for manufacturing activated carbon for air adsorbents where thickness is important. In one embodiment, the viscose rayon may be manufactured by extracting cellulose fibers from wood pulp, such as jutes, pine, beech, or eucalyptus.

In one embodiment, the air adsorbent may include the activated carbon fabric, which is wound at least once to define a hollow roll shape. Since the activated carbon fabric has a roll shape, the air adsorbent may be easily disposed in a large-volume sound resonance space, enabling reduced processing time and cost, and preventing damage to the air adsorbent during the manufacturing process. Additionally, since the activated carbon fabric has a shape similar to a hollow pipe, air may easily flow in and out of the activated carbon fabric, improving its air permeability.

In this embodiment, the air adsorbent may further include an adhesive member disposed at an end of the roll-shaped activated carbon fabric to maintain the roll shape. More specifically, the activated carbon fabric may include one end that defines an inner surface of the roll-shaped activated carbon fabric and the other end that defines an outer surface of the roll-shaped activated carbon fabric. The adhesive member may be disposed on an inner surface of the other end, allowing the other end to adhere to and be fixed to another portion of the activated carbon fabric.

The adhesive member may have a higher air permeability than the activated carbon fabric and may have a mesh or a web shape. When the air permeability of the adhesive member is lower than that of the activated carbon fabric, it may hinder airflow, potentially reducing the performance of the air adsorbent.

The adhesive member may be made of polyester (PES), polyamide (PA), thermoplastic polyurethane (TPU), or ethylene-vinyl acetate (EVA), and may have an adhesive resin applied to it. Using these materials, a mesh or web shape may be manufactured by melt-blown or electrospinning methods. The adhesive resin may be a mixture of acrylate or silicone resin with ester rubber, phenolic resin, polyisobutylene, castor oil, and others.

In one embodiment, the adhesive member may have a mass per unit area ranging from about 15 g/m² to about 25 g/m², a melting point ranging from about 100° C. to about 120° C., a specific gravity ranging from about 1 g/cm³ to about 1.1 g/cm³, a melt index ranging from about 5 cm³/10 min to about 500 cm³/10 min, and heat press condition ranging from about 120° C. to about 150° C. for about 10 to about 15 seconds.

The pouch member may store the activated carbon fabric thereinside, facilitating the manufacturing process and preventing damage to the activated carbon fabric. The pouch member may have air permeability ranging from about 200 cm³/cm²/sec to about 550 cm³/cm²/sec, and a thickness ranging from about 0.07 mm to about 0.15 mm.

In one embodiment, the pouch member may be made of polymer resins such as PET (polyethylene terephthalate) or PLA (polylactic acid), and may be nonwoven fabric, mesh, web, or film with micro-holes allowing air molecules to pass through. Here, the nonwoven fabric shape may be preferable for its excellent air permeability while also preventing foreign substances generated from the activated carbon fabric from being discharged to the outside.

### Method for Manufacturing the Activated Carbon Fabric

A method for manufacturing the activated carbon fabric includes a step of preparing cellulose fibers, a step of manufacturing a cellulose fabric using the cellulose fibers, and a step of carbonizing the cellulose fabric.

The cellulose fibers may be natural cellulose fibers, such as cotton or linen, or synthetic cellulose fibers, such as viscose rayon, modal, or lyocell, with viscose rayon being preferable. In one embodiment, the cellulose fibers may be viscose rayon made by extracting cellulose fibers from wood pulps, such as jutes, pine, beech, or eucalyptus.

Next, the step of manufacturing the cellulose fabric may include weaving the cellulose yarns, which are made by the cellulose fibers. In one embodiment, the cellulose fibers may be plied or twisted into yarns and then woven to manufacture the fabric. The weaving method may be a twill weaving method.

Next, the carbonizing step may include a step of heating the cellulose fabric at least once. In one embodiment, the cellulose fabric may be disposed in a carbonizer and heated at a first temperature, then further heated to a second temperature that is higher than the first temperature. As an example, the first temperature may range from about 150° C. to about 400° C., and the second temperature may range from about 400° C. to about 950° C. Additionally, the time for heating to the first temperature may range from about 15 minutes to about 1 hour, and the time for heating to the second temperature may range from about 15 minutes to about 35 minutes. Since performing the low-temperature carbonization process for a longer time, an activated carbon fabric with improved structural integrity may be obtained. Through this, the activated carbon fabric with excellent specific surface area characteristics may be manufactured.

### Method for Manufacturing Air Adsorbent for Speakers

A method for manufacturing an air adsorbent for speakers according to an embodiment of the present invention includes a step of preparing the activated carbon fabric; a step of rolling the activated carbon fabric, having one end rolled inward to define a hollow roll shape; a step of fixing the other end of the activated carbon fabric using an adhesive member; and a step of cutting the roll-shaped activated carbon fabric along a longitudinal direction of the other end of the activated carbon fabric. In one embodiment, after the step of cutting, the method may further include a step of inserting the cut roll-shaped activated carbon fabric into an air-permeable pouch and sealing the air-permeable pouch.

The step of preparing the activated carbon fabric may be as described earlier.

The step of manufacturing the roll-shaped activated carbon fabric may include a step of rolling the activated carbon fabric. In this case, the activated carbon fabric may have a hollow structure, making it similar in shape to a pipe with an inner surface located inside the hollow and an outer surface exposed to the outside. One end of the activated carbon fabric may be disposed on the inner surface of the activated carbon fabric, and the other end may be disposed on the outer surface of the activated carbon fabric. In one embodiment, one end of the activated carbon fabric may be attached to a cylindrical rod to wrap the cylindrical rod up to create a spiral-roll shape, and an adhesive member, which will be described later, may be applied to the other end of the activated carbon fabric. Afterward, the cylindrical rod may be removed, leaving the hollow roll shape.

The step of fixing the other end of the activated carbon fabric using the adhesive member may ensure that the roll shape remains intact without unrolling. The adhesive member may be disposed inside the other end, which is disposed on the outer surface of the activated carbon fabric, to attach and fix the other end to a surface in contact with the inside the other end of the activated carbon fabric. The adhesive member may have heat press condition ranging from about 120° C. to about 150° C. for about 10 seconds to about 15 seconds, and the other end of the activated carbon fabric may be heated and pressed from the outside to adhere.

The step of cutting the roll-shaped activated carbon fabric along the longitudinal direction of the other end may be a step of processing the activated carbon fabric into an appropriate size. For the efficiency of the process, a long-activated carbon fabric may be rolled into a roll shape and then cut at regular intervals along the longitudinal direction thereof. The cut activated carbon fabric may have a relatively shorter roll shape.

Next, after the cut roll-shaped activated carbon fabric is inserted into an air-permeable pouch, the step of sealing the air-permeable pouch may be performed.

### Method for Manufacturing Air Adsorbent for Speakers

A method for manufacturing an air adsorbent for speakers according to another embodiment of the present invention includes a step of preparing a plurality of activated carbon fabrics manufactured as described above; a step of placing and fixing a mesh or web-shaped adhesive member on one activated carbon fabric; a step of placing another activated carbon fabric on the adhesive member to manufacture a laminate structure; a step of fixing the laminate structure; and a step of cutting the fixed laminate structure along a lamination direction. In one embodiment, after the step of cutting the fixed laminate structure along the lamination direction, the method may further include a step of inserting the cut roll-shaped activated carbon fabric into an air-permeable pouch and sealing the air-permeable pouch.

The step of preparing the activated carbon fabric is as described earlier.

The step of disposing and fixing a mesh or web-shaped adhesive member on one the activated carbon fabric and the step of disposing another activated carbon fabric on the adhesive member to manufacture the laminate structure may be performed once or more than twice, thereby manufacturing the laminate structure in which the activated carbon fabric and the adhesive member are sequentially laminated. In one embodiment, by further performing the step of disposing another activated carbon fabric on the adhesive member, the activated carbon fabric may be disposed at both the topmost and bottommost layers of the laminate structure.

This step may further include a step of pressing the laminate structure, or a step of heating and pressing the laminate structure. This step may be performed each time the activated carbon fabric or the adhesive member is placed and may be performed after completing the laminating process. This step may be performed using a heat press device or the like and may be carried out at a temperature of about 120° C. to about 150° C. and a pressure of about 0.5 MPa to about 1.0 MPa for 10 seconds to 15 seconds.

In this step, the adhesive member is the same as previously described and has higher air permeability than the activated carbon fabric. The adhesive member may have a mesh or web shape. The adhesive member may be provided smaller than the laminate structure to prevent exposure from the side surface of the laminate structure.

The step of cutting the fixed laminate structure in the lamination direction involves cutting the laminate structure at least once from an upper end of the laminate structure toward a lower end of the laminate structure to manufacture a plurality of cut laminate structures. Through this process, an air adsorbent suitable for use in speakers with a uniform and appropriate size may be manufactured.

This step may be performed using a laser cutter or a high-speed cutting machine.

In the step of inserting the cut laminate structure into the air-permeable pouch and sealing the air-permeable pouch, as described above, the air-permeable pouch may have a shape of a nonwoven fabric, mesh, web, or a film with micro-holes that allow air molecules to pass through. The method of sealing the air-permeable pouch may be performed by adhesion using an adhesive or by heating and pressing.

### Manufacturing Example: Manufacturing of Air Adsorbent

Manufacturing Example 1: A cellulose fabric was manufactured by twill weaving using viscose rayon manufactured from wood pulp. The fabric was then heated at a maximum temperature of about 400° C. for 1 hour, followed by heating at a maximum temperature of about 950° C. for 35 minutes to manufacture an air adsorbent.

Manufacturing Example 2: The process was performed in the same manner as in example 1, except for using plain weaving in the manufacturing of the cellulose fabric.

Manufacturing Example 3: The process was performed in the same manner as in example 1, except for using double weaving in the manufacturing of the cellulose fabric.

Manufacturing Example 4: Viscose rayon manufactured using wood pulp was crushed into multiple strands, evenly dispersed, and pressed to manufacture a nonwoven air adsorbent.

Manufacturing Example 5: Fibers made of phenol-aldehyde resin, which is a cross-linked novolac resin, were used as a precursor. The fibers were twill woven, subjected to a stabilization process through oxidation at about 200° C., and then heated at about 1,100° C. to manufacture an air adsorbent.

### Experimental Example: Air Permeability Analysis of Air Adsorbent

The air permeability of examples 1 to 5 was measured according to the Frazier method defined in ISO 9237:1995. The measured air permeability is shown in table 1. As a result of the measurement, the air adsorbent manufactured by twill weaving exhibited the highest air permeability, and the air adsorbent manufactured from cellulose fibers showed significantly superior air permeability compared to that manufactured from phenol fibers.

TABLE 1

| Classification | Air permeability (cm3/cm2/s) |
|---|---|
| Manufacturing Example 1 | 77.8 |
| Manufacturing Example 2 | 57.4 |
| Manufacturing Example 3 | 25.1 |
| Manufacturing Example 4 | 30.6 |
| Manufacturing Example 5 | 7.4 |

### Embodiment: Manufacturing of Speaker-Box System

Embodiment 1 A speaker device with a sound resonance space having a volume of about 218 cc, a diameter of about 80 mm, and a nominal impedance of about 8Ω was prepared. The air adsorbent of manufacturing example 1 was cut to a volume of about 100 cc and then inserted into the sound resonance space, followed by sealing.

Embodiment 2 The method was carried out in the same way as embodiment 1, except for the use of the air adsorbent of manufacturing example 2.

Embodiment 3 The method was carried out in the same way as embodiment 1, except for the use of the air adsorbent of manufacturing example 3.

Comparative Example 1: The speaker device was used as it is without using an air adsorbent.

### Experimental Example: Analysis of Speaker Performance

The resonance frequency and the change in resonance frequency of examples 1 to 3 and comparative example 1 were measured. The measurement method involves placing the air adsorbents of the embodiments and comparative examples into the sound resonance space of the speaker-box system, completely sealing the sound resonance space to prevent airflow, applying a variable frequency voltage to the speaker-box system, and confirming the impedance-frequency resonance through an impedance meter. The impedance values for each frequency were then recorded. The results of measurements are shown in Table 2. The measurement results showed that in the embodiments, the change in resonance frequency was the highest when using the air adsorbent made with twill weaving. Additionally, the change in resonance frequency for the embodiment using the air adsorbent made from cellulose fibers was significantly higher compared to comparative example 2.

TABLE 2

| | | | Change in |
|---|---|---|---|
| | | Resonant | resonant |
| | | frequency | frequency |
| Classification | Air adsorbent | (Fo) (Hz) | (ΔFo) (Hz) |
| Comparative | None | 233.2 | 0 |
| Example 1 | | | |
| Embodiment 1 | Manufacturing example 1 | 215.8 | 17.4 |
| Embodiment 2 | Manufacturing example 2 | 220.6 | 12.6 |
| Embodiment 3 | Manufacturing example 3 | 230.5 | 2.7 |

### Embodiment Manufacturing of Speaker-Box System Using Air Adsorbent Including Pouch

Embodiment 4: A speaker device with a sound resonance space having a volume of about 218 cc, a diameter of about 80 mm, and a nominal impedance of about 8Ω was prepared. The air adsorbent was prepared by inserting the activated carbon fabric of manufacturing example 1, which is cut to a volume of 100 cc, into a pouch made of PET film having an air-permeable hole with air permeability of about 510 cm³/cm²/s and sealing edges of the pouch. The air adsorbent was inserted into the sound resonance space and sealed.

Embodiment 5: The manufacturing process was performed in the same manner as in example 1, except for using a pouch with an air permeability of about 461 cm³/cm²/s.

Embodiment 6 The manufacturing process was performed in the same manner as in example 1, except for using a pouch with an air permeability of about 278 cm³/cm²/s.

Embodiment 7 The manufacturing process was performed in the same manner as in example 1, except for using a pouch with an air permeability of about 79 cm³/cm²/s.

Embodiment 8 The manufacturing process was performed in the same manner as in example 1, except for using a pouch with an air permeability of about 12 cm³/cm²/s.

Comparative Example 2: The speaker device was used as it is without using an air adsorbent.

### Experimental Example: Analysis of the Speaker Performance

The resonance frequency and resonance frequency variation of the speaker were measured for embodiments 4 to 8 and comparative example 3. The measurement results are shown in table 3. The measurement results showed that embodiments 4 to 6, which used pouches with an air permeability of about 278 to about 510 cm³/cm²/s, exhibited the same characteristics as embodiment 1, which did not use a pouch. However, examples 7 and 8, which used pouches with lower air permeability, showed a relatively smaller resonance frequency variation.

TABLE 3

| | | | Change in |
|---|---|---|---|
| | Pouch air | Resonant | resonant |
| | permeability | frequency | frequency |
| Classification | (cm3/cm2/s) | (Fo) (Hz) | (ΔFo) (Hz) |
| Comparative Example 2 | — | 233.2 | 0 |
| Embodiment 4 | 510 | 215.8 | 17.4 |
| Embodiment 5 | 461 | 215.8 | 17.4 |
| Embodiment 6 | 278 | 215.8 | 17.4 |
| Embodiment 7 | 79 | 218.5 | 14.7 |
| Embodiment 8 | 12 | 235.7 | (−)2.5 |

The air adsorbent and the sound equipment including the same according to an embodiment of the present invention may improve sound quality.

Additionally, according to the present invention, the low-frequency characteristics of speaker-box systems for televisions, which have a large back volume and high output, may be improved.

According to the present invention, the issue of foreign substances, such as dust, generated from conventional air adsorbents in the form of particles, granules, or powder may be minimized.

The present invention is not limited to the above-described embodiments and the accompanying drawings but is defined by the appended claims. Accordingly, various substitutions, modifications, and changes may be made by those of ordinary skill in the art without departing from the technical spirit of the invention as set forth in the claims, and such variations shall also fall within the scope of the present invention.

What is claimed is:

1. An air adsorbent for a speaker, comprising:
an activated carbon fabric comprising activated carbon fibers manufactured by carbonizing a cellulose fabric containing cellulose fibers,
wherein the cellulose fabric is manufactured by weaving bundles of the cellulose fibers, wherein each bundle is provided by twisting the plurality of cellulose fibers,
the activated carbon fabric has a thickness of about 0.3 mm to about 0.9 mm, an air permeability of about 60 cm³/cm²/s to about 90 cm³/cm²/s, and a BET-specific surface area of about 1000 m²/g to about 1500 m²/g, and
each of the activated carbon fibers has pores, each of which has an average diameter of about 1 nm to about 5 nm.

2. The air adsorbent of claim 1, wherein a benzene gas has adsorption capacity of about 40 wt % to about 50 wt % under a condition of 20° C.

3. The air adsorbent of claim 1, wherein the activated carbon fabric has a weight per area of about 120 g/m² to about 170 g/m².

4. The air adsorbent of claim 1, wherein the cellulose fabric is woven using twill weaving.

5. The air adsorbent of claim 1, further comprising:
the activated carbon fabric of claim 1; and
an adhesive member configured to adhere surfaces of the activated carbon fabric to maintain a predetermined shape,
wherein the activated carbon fabric has a roll shape with a hollow defined by being wound at least once and includes one end providing an inner surface of the roll shape and the other end providing an outer surface of the roll shape, and
the adhesive member is disposed on an inner surface of the other end to allow the other end of the activated carbon fabric to adhere to another portion of the activated carbon fabric to be fixed.

6. The air adsorbent of claim 5, wherein the adhesive member has an air permeability of about 500 cm³/cm²/s to about 2000 cm³/cm²/s and has a nonwoven fabric shape.

7. The air adsorbent of claim 1, further comprising a pouch member that encloses the activated carbon fabric,
wherein the pouch member has an air permeability of about 200 cm³/cm²/s to about 550 cm³/cm²/s and a thickness of about 0.07 mm to about 0.15 mm.

8. Sound equipment comprising the air adsorbent for the speaker of claim 1.

9. A method for manufacturing an air adsorbent for a speaker, the method comprising:
preparing the activated carbon fabric;
rolling the activated carbon fabric so that one end of the activated carbon fabric is disposed on an inner surface thereof to form a hollow roll shape; fixing the other end of the activated carbon fabric using an adhesive member; and
cutting the roll-shaped activated carbon fabric along a longitudinal direction of the other end of the activated carbon fabric.

10. The method of claim 9, further comprising, after the cutting of the roll-shaped activated carbon fabric:
inserting the cut roll-shaped activated carbon fabric into an air-permeable pouch; and
sealing the air-permeable pouch.

11. A method for manufacturing an air adsorbent for a speaker, the method comprising:
preparing the plurality of activated carbon fabrics of claim 1;
placing and fixing a mesh or web-shaped adhesive member on one activated carbon fabric;
placing another activated carbon fabric on the adhesive member to manufacture a laminate; and
cutting the fixed laminate in a lamination direction.

12. The method of claim 11, further comprising, after the cutting of the fixed laminate in the lamination direction:
inserting the cut laminate into an air-permeable pouch; and
sealing the air-permeable pouch.
